(19) United States
(12) Patent Application Publication
(10) Pub. No.: US 2015/0260891 A1
(43) Pub. Date: Sep. 17, 2015

(54) Cool Blue Welding Lens

(71) Applicant: Wendell G Poplin, Kountze, TX (US)

(72) Inventor: Wendell G Poplin, Kountze, TX (US)

(21) Appl. No.: 14/657,775

(22) Filed: Mar. 13, 2015

Related U.S. Application Data
(60) Provisional application No. 61/953,245, filed on Mar. 14, 2014.
(60) Provisional application No. 61/983,890, filed on Apr. 24, 2014.

(51) Int. Cl. G02B 5/28; G02C 7/10; A61F 9/06

(52) U.S. Cl. CPC G02B 5/285; A61F 9/065; G02C 7/107; G02C 7/104; G02C 2202/16

(57) ABSTRACT

An apparatus for transmitting favorable light from a welding arc to the eyes of the welder contains a dichroic filter, an adhesive, and a welding lens. The dichroic filter is superimposed onto the welding lens with the adhesive. More specifically, when considering a cross section, the adhesive is positioned in between the dichroic filter and the welding lens. In order to utilize the apparatus during a welding process, the apparatus is positioned within the viewing window of a welding helmet. In particular, the dichroic filter is oriented outwards such that the welding lens is positioned in close proximity to the eyes of the welder when the welding helmet is worn. The apparatus provides the welder a satisfying welding experience and also protects the eyes of the welder from harmful ultraviolet light and infrared light.

The current application claims a priority to the U.S. Provisional Patent application Ser. No. 61/953,245 filed on Mar. 14, 2014. The current application claims a priority to the U.S. Provisional Patent application Ser. No. 61/983,890 filed on Apr. 24, 2014.

### FIELD OF THE INVENTION

The present invention relates generally to welding safety accessories. More specifically, the present invention is a dual-layered welding lens with a conventional welding lens and dichroic filter lens. The dual-layered welding lens is capable of transmitting a favorable blue light while reflecting all other light.

### BACKGROUND OF THE INVENTION

Welding is a process of joining materials that involves the melting of work pieces that are then joined via a joint formed from a pool of molten filler material that is allowed to cool to form the joint. Welding is an often dangerous process due to the nature of the equipment and materials involved. Many common welding procedures expose the welder to an open electric arc or flame that poses a significant risk of burns from heat and sparks. The welder typically dons protective clothing such as specially designed long-sleeved jackets and gloves to avoid direct exposure to extreme heat. In addition to the dangers posed by heat, welding often exposes the welder to particulate matter and gases produced during the welding process that are extremely harmful if inhaled. As a result, proper ventilation in a workspace is required due to the toxicity of the fumes produced during welding. An additional danger posed by welding is present in the form of eye damage caused by exposure of the eyes to ultraviolet light. Eye damage may be caused in the form of inflammation of the cornea and burning of the retina. A welder often wears a welding helmet that is designed to cover the face and neck for protection from heat, sparks, and ultraviolet light. A welding helmet typically features a viewing window that is covered with a lens that is capable of filtering ultraviolet light emitted during welding. More recent welding helmets may also include modern features such as self-darkening lenses, as well as fans for both cooling the welder and flushing toxic fumes away from the welder's face. The present invention seeks to enhance and improve upon conventional welding safety accessories.

The present invention is a dual-layered welding lens featuring a conventional welding lens as well as a dichroic filter lens. In the preferred embodiment of the present invention, the conventional welding lens forms the inner layer of the dual-layered welding lens while the dichroic filter lens forms the outer layer. The two layers of the dual-layered welding lens are joined via an adhesive such as liquid optically-clear adhesive (LOCA). An adhesive such as LOCA is favorable due to the lack of condensation and fogging produced, resistance to extreme temperatures, optical clarity, and its liquid properties that make the adhesive ideal for filling in gaps between two surfaces. The dual-layered welding lens may be installed into the viewing window of a conventional welding helmet. The dichroic filter lens of the dual-layered welding lens is capable of filtering colors of light emitted during welding, and is made of borosilicate glass which has ultraviolet light filtering capabilities and is much stronger than most other glasses. In the preferred embodiment of the present invention, the dichroic filter lens is capable of transmitting blue light, which has a wavelength in the range of 450 nm-530 nm, while reflecting other wavelengths including certain ultra violet light and infrared light. Blue light is highly favored by welders due to the lower intensity of the glare associated with the welding arc. Moreover, the blue light reduces the strain on the eyes which usually occurs due to the inability of the eyes to adjust to bright light. In the preferred embodiment of the present invention, the conventional welding lens comprises a gold mirror welding lens that is capable of reflecting ultraviolet light while absorbing certain wavelengths. Additionally, because the frequency of visible blue light is very near to ultraviolet range on the electromagnetic spectrum, the conventional welding lens is able to reflect ultraviolet light emitted during welding. Overall, the assembly of the dichroic filter and the welding lens has the ability to absorb certain wavelengths and also reflect the remaining portion of wavelengths that can be harmful to the human eye. The present invention transmits a blue light which is preferred by many welders in the welding industry.

### BRIEF DESCRIPTION OF THE DRAWINGS

FIG. 1 is a perspective view of a welding helmet, wherein the present invention is positioned within the viewing window of the welding helmet.

FIG. 2 is an exploded perspective view illustrating the positioning of the present invention within the welding helmet.

FIG. 3 is an exploded side view illustrating the positioning of the present invention within the welding helmet.

FIG. 4 is a cross-sectional side view illustrating the present invention, wherein a thin film is in between a substrate and an adhesive.

FIG. 5 is a cross sectional side view illustrating the present invention, wherein the substrate is in between the thin film and the adhesive.

FIG. 6 is a ray diagram illustrating the path of light waves from the welding arc to the eyes of the welder.

### DETAIL DESCRIPTIONS OF THE INVENTION

All illustrations of the drawings are for the purpose of describing selected versions of the present invention and are not intended to limit the scope of the present invention.

The present invention introduces an apparatus that transmits favorable light to the eyes of a welder during a welding process. The favorable light transmitted by the present invention eliminates the harmful effects of ultraviolet light and also provides the welder a pleasant welding experience.

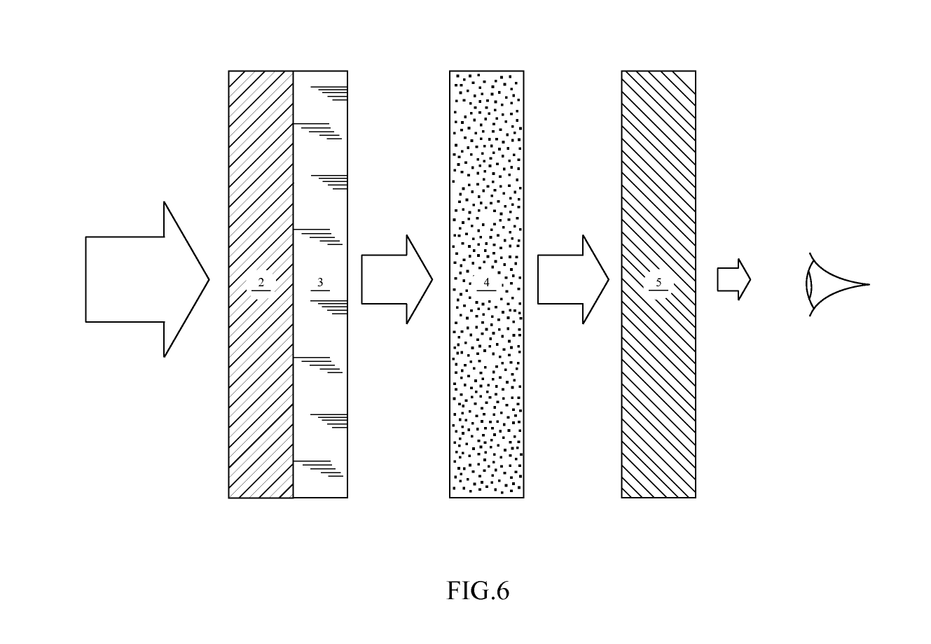

The present invention comprises a dichroic filter 1, an adhesive 4, and a welding lens 5. The dichroic filter 1 reflects most light frequencies emitted from a welding process and allows certain light frequencies to pass through. In the preferred embodiment of the present invention, the dichroic filter 1 transmits wavelengths that help create a blue light as a final result. In particular, the dichroic filter 1 in the preferred embodiment of the present invention transmits wavelengths between 450 nm and 530 nm on the electromagnetic spectrum. However, in another embodiment of the present invention the dichroic filter 1 can be different in order to transmit different light frequencies to the eyes of the welder. The light transmitted through the dichroic filter 1 is further filtered by the welding lens 5 through absorption. The welding lens 5 restricts light from passing through and provides a truer color palette to the eyes of the welder. FIG. 6 illustrates light waves travelling through the dichroic filter 1 and the welding lens 5. In the preferred embodiment of the present invention, a gold mirror welding lens is utilized as the welding lens 5. As a result, a blue light is seen by the welder after the light waves travel through both the dichroic filter 1 and the welding lens 5. The dichroic filter 1 is positioned adjacent the welding lens 5 with the adhesive 4. In particular, the adhesive 4 is utilized to superimpose the dichroic filter 1 onto the welding lens 5. In order to do so, the adhesive 4 is positioned in between the dichroic filter 1 and the welding lens 5.

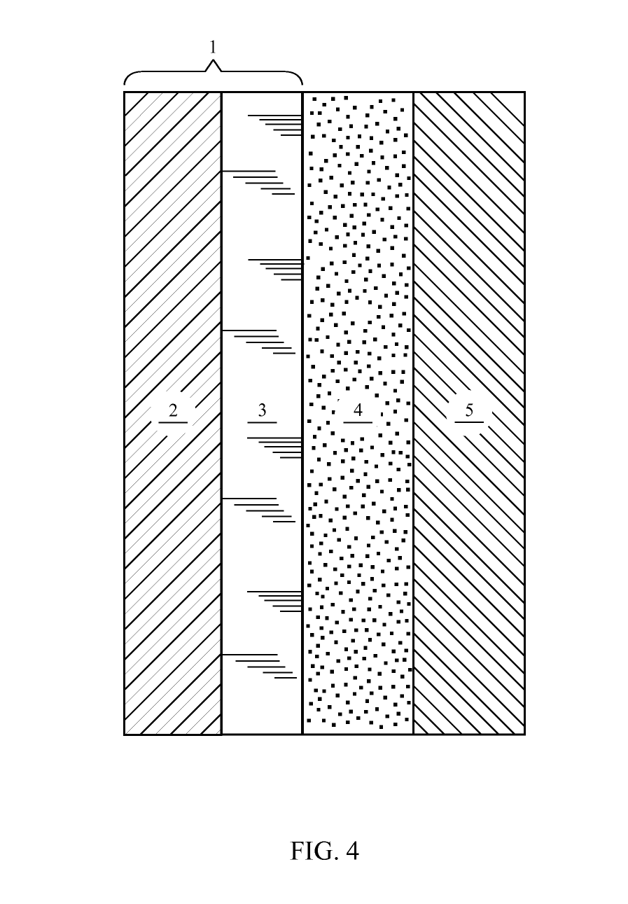

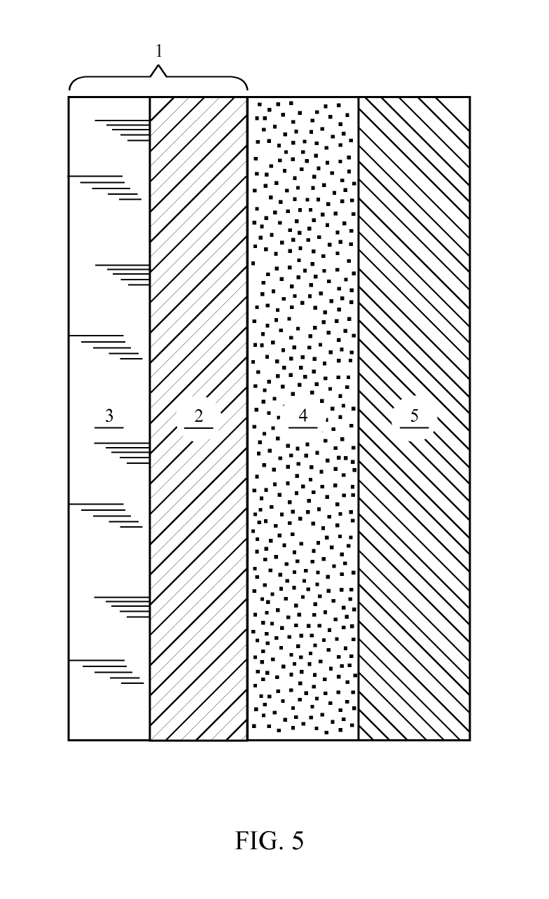

As seen in FIG. 4 and FIG. 5, the dichroic filter 1 comprises a substrate 2 and a thin film 3. In the preferred embodiment of the present invention, the substrate 2 is made of borosilicate, whereas the thin film 3 is a metal which is evaporated onto the substrate 2. In particular, the thin film 3 is evaporated in a perfect vacuum and in subzero temperatures to eliminate any foreign particles from being in between the substrate 2 and the thin film 3. As a result of the evaporation process, the thin film 3 is superimposed onto the substrate 2. Since the substrate 2 is made of borosilicate, and the thin film 3 is evaporated onto the substrate 2, the dichroic filter 1 is able to withstand high temperatures that occur during a welding process. The dichroic filter 1 reflects certain light wavelengths, such that only the preferred wavelengths are transmitted through to the eyes of the welder.

In the preferred embodiment of the present invention, the dichroic filter 1 is oriented such that the thin film 3 is positioned in between the substrate 2 and the adhesive 4. FIG. 4 illustrates the thin film 3 being positioned in between the substrate 2 and the adhesive 4. As a result of the orientation, the thin film 3 is not exposed to the environment which is essential for the longevity of the dichroic filter 1. However, in another embodiment of the present invention, the substrate 2 can be positioned in between the thin film 3 and the adhesive 4 as illustrated in FIG. 5. In such instances, the thin film 3 is the outermost layer of the present invention. Regardless of the orientation, the dichroic filter 1 reflects certain wavelengths and transmits the desired wavelengths through to the welding lens 5.

As mentioned earlier, the dichroic filter 1 is superimposed onto the welding lens 5 with the adhesive 4. In the preferred embodiment of the present invention, liquid optically clear adhesive (LOCA) is utilized as the adhesive 4. LOCA is utilized since LOCA is able to maintain its adhesive properties at high temperatures and also prevents fogging which is beneficial to the welder. Even though LOCA is utilized in the preferred embodiment of the present invention, another comparable adhesive which transmits light similarly can be utilized in other embodiments of the present invention. The ability to transmit light with minimum refraction and reflection is essential to transmit the desired light waves to the eyes of the welder. In addition to superimposing the dichroic filter 1 onto the welding lens 5, the adhesive 4 also seals the space in between the dichroic filter 1. Sealing the air gap in between the dichroic filter 1 and the welding lens 5 is important to provide an unobstructed path for the light waves to travel through. More specifically, any air gaps in between the dichroic filter 1 and the welding lens 5 may result in refraction, which may reduce the number of light waves transmitted to the eyes of the welder. Therefore, as illustrated in FIG. 4 and FIG. 5, the adhesive 4 is layered onto the surface area of both the dichroic filter 1 and the welding lens 5 such that the gap in between the dichroic filter 1 and the welding lens 5 is completely sealed. The dichroic filter 1 and the welding lens 5 utilized in the preferred embodiment of the present invention are rectangular in shape. Therefore, when the dichroic filter 1 is superimposed onto the welding lens 5 with the adhesive 4, the four edges of the dichroic filter 1 are aligned with the four edges of the welding lens 5.

A majority of harmful light waves are reflected by the gold mirror lens utilized in the preferred embodiment of the present invention. Additionally, the gold mirror lens is scratch-resistant and resistant to radiant light when the welding lens 5 is positioned adjacent to the dichroic filter 1. Being scratch-resistant is important for the longevity of the present invention. On the other hand, being resistant to radiant light is important to eliminate thermal radiation. In another embodiment of the present invention, a dark lens can be utilized as the welding lens 5. Similar to the preferred embodiment of the present invention, the dichroic filter 1 is superimposed onto the dark lens with the adhesive 4.

In another embodiment of the present invention, an opacity adjusting lens can be utilized as the welding lens 5. In particular, the opacity of the welding lens 5 adjusts according to the light emitted from the welding arc. A plurality of light intensity sensors and a portable power source can be utilized to control the opacity adjusting lens. Each of the plurality of light intensity sensors can be, but is not limited to, a light dependant resistor. If light dependent resistors are utilized as the plurality of light intensity sensors, then the light dependent resistors detect the brightness of the welding process and adjust the resistance accordingly. As a result, the voltage applied across the opacity adjusting lens alternates such that the opacity of the opacity adjusting lens changes.

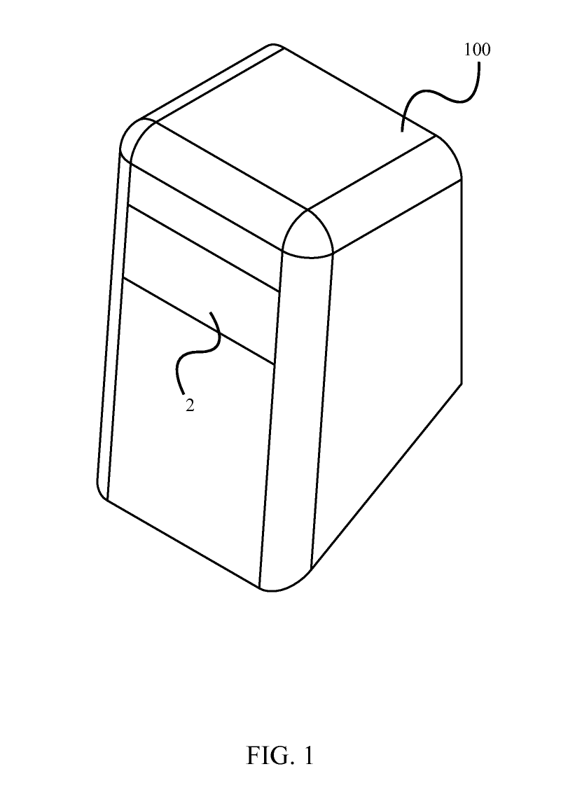

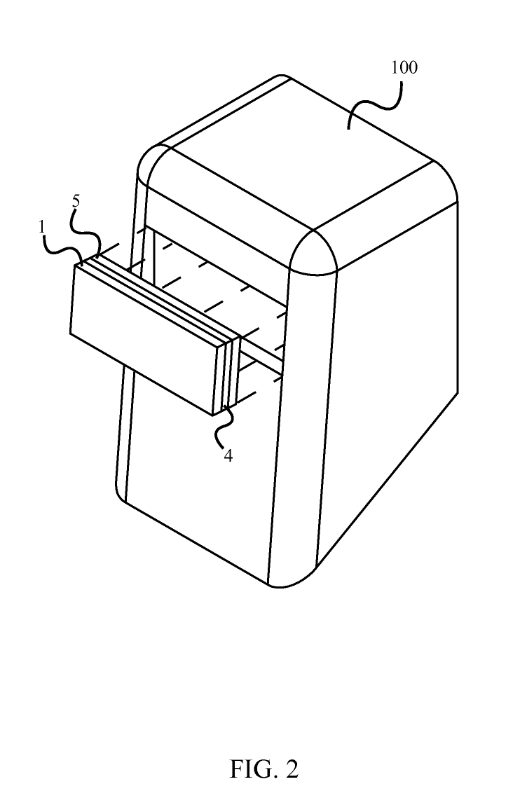

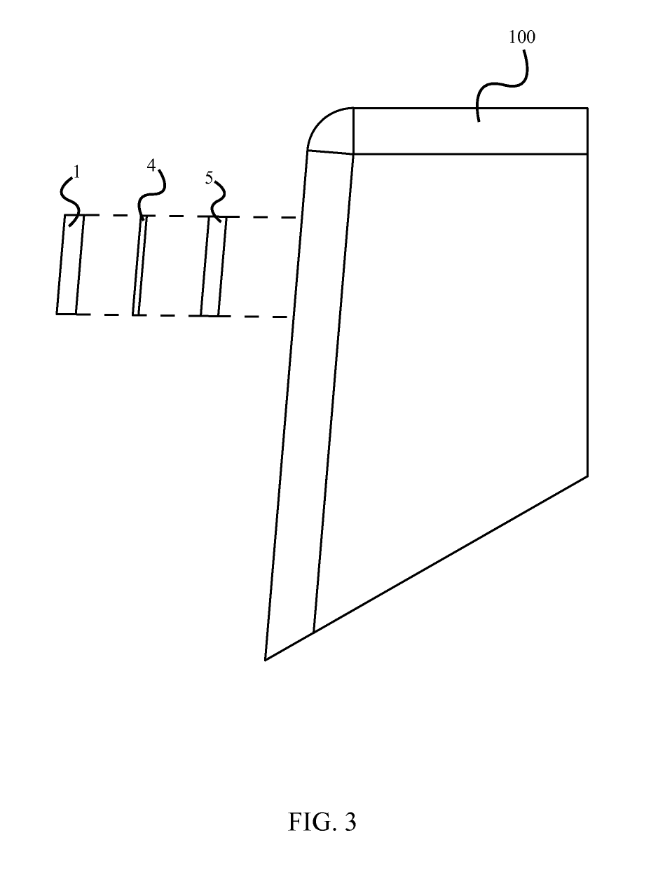

When utilizing the present invention, the following process is followed. As an initial step, the present invention is placed within a viewing window of the welding helmet 100 as seen in FIG. 1-3. In doing so, the present invention is oriented such that the dichroic filter 1 is placed facing outwards. In particular, the welding lens 5, which is the gold minor welding lens 5 in the preferred embodiment of the present invention, is placed internally and close to the eyes of the welder. When the present invention is utilized during the welding process, light waves are emitted towards the dichroic filter 1. When the light waves reach the dichroic filter 1, certain wavelengths are reflected while specific wavelengths are transmitted through the dichroic filter 1. The light waves that travel through the dichroic filter 1 proceed to travel through the adhesive 4 towards the welding lens 5. Upon reaching the welding lens 5, a majority of the waves are reflected and waves of a specific wavelength travel through the welding lens 5. In the preferred embodiment of the present invention, wavelengths corresponding to blue light are transmitted through the welding lens 5 towards the eyes of the welder. More specifically, wavelengths within the range of 450 nanometers-530 nanometers are transmitted towards the eyes of the welder.

Although the invention has been explained in relation to its preferred embodiment, it is to be understood that many other possible modifications and variations can be made without departing from the spirit and scope of the invention as hereinafter claimed.

What is claimed is:

1. A dual-layered welding lens for transmitting favorable light comprises:
a dichroic filter;
an adhesive;
a welding lens;
the dichroic filter comprises a substrate and a thin film;
the dichroic filter being superimposed onto the welding lens by the adhesive;
the adhesive being positioned in between the dichroic filter and the welding lens; and
the thin film being superimposed onto the substrate.

2. The dual-layered welding lens for transmitting favorable light as claimed in claim 1, wherein the thin film is positioned in between the substrate and the adhesive.

3. The dual-layered welding lens for transmitting favorable light as claimed in claim 1, wherein the substrate is positioned in between the thin film and the adhesive.

4. The dual-layered welding lens for transmitting favorable light as claimed in claim 1, wherein the adhesive layer is a liquid optically clear adhesive.

5. The dual-layered welding lens for transmitting favorable light as claimed in claim 1, wherein the welding lens is a gold mirror lens.

6. The dual-layered welding lens for transmitting favorable light as claimed in claim 1, wherein the welding lens is a dark lens.

7. The dual-layered welding lens for transmitting favorable light as claimed in claim 1, wherein the welding lens is an automatic opacity adjusting lens.

8. A dual-layered welding lens for transmitting favorable light comprises:
a dichroic filter;
an adhesive;
a welding lens;
the dichroic filter comprises a substrate and a thin film;
the dichroic filter being superimposed onto the welding lens by the adhesive;
the adhesive being positioned in between the dichroic filter and the welding lens;
the thin film being superimposed onto the substrate; and
the thin film being positioned in between the substrate and the adhesive.

9. The dual-layered welding lens for transmitting favorable light as claimed in claim 8, wherein the adhesive layer is a liquid optically clear adhesive.

10. The dual-layered welding lens for transmitting favorable light as claimed in claim 8, wherein the welding lens is a gold mirror lens.

11. The dual-layered welding lens for transmitting favorable light as claimed in claim 8, wherein the welding lens is a dark lens.

12. The dual-layered welding lens for transmitting favorable light as claimed in claim 8, wherein the welding lens is an automatic opacity adjusting lens.

13. A dual-layered welding lens for transmitting favorable light comprises:
a dichroic filter;
an adhesive;
a welding lens;
the dichroic filter comprises a substrate and a thin film;
the dichroic filter being superimposed onto the welding lens by the adhesive;
the adhesive being positioned in between the dichroic filter and the welding lens;
the thin film being superimposed onto the substrate; and
the welding lens being a gold mirror lens.

14. The dual-layered welding lens for transmitting favorable light as claimed in claim 13, wherein the thin film being positioned in between the substrate and the adhesive.

15. The dual-layered welding lens for transmitting favorable light as claimed in claim 13, wherein the substrate being positioned in between the thin film and the adhesive.

16. The dual-layered welding lens for transmitting favorable light as claimed in claim 13, wherein the adhesive layer is a liquid optically clear adhesive.
